(19) United States
(12) Patent Application Publication
(10) Pub. No.: US 2009/0253814 A1
(43) Pub. Date: Oct. 8, 2009

(54) COMPACT REACTOR

(76) Inventors: Nicole Schodel, Munchen (DE); Bruno Nowakowski, Engelsberg (DE); Thomas Hecht, Gauting (DE); Wolfgang Muller, Munchen (DE); Herbert Aigner, Engelsberg (DE); Stephanie Neuendorf, Hohenschaftlarn (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/418,097

(22) Filed: Apr. 3, 2009

(30) Foreign Application Priority Data
Apr. 4, 2008 (DE) 10 2008 017 342.8

(51) Int. Cl. C07C 27/06; B01J 35/02

(52) U.S. Cl. 518/700; 422/212

(57) ABSTRACT

The invention relates to a compact reactor that consists of a number of plates that are arranged like stacks and spaced some distance apart, whereby
a) The plates are separated by spacers and are sealed off from one another in a gas-tight manner,
b) The plates are profiled in the shape of waves, so that flow channels are formed by the wave troughs that are separated from one another by the wave crests (fins),
c) The flow channels run parallel to one another and parallel to one side of the plate,
d) The flow channels at least partially contain at least one catalyst material that is introduced such that gaseous and/or liquid media can flow through the flow channels, and
e) The compact reactor has means (headers) for feeding at least two gaseous and/or liquid media to the flow channels or removing them therefrom.
   The invention also relates to the use of a compact reactor. The gaseous and/or liquid medium that flows in the flow channels can flow through the fins between the individual flow channels of a plate.

### SUMMARY OF THE INVENTION

The invention relates to a compact reactor comprising a number of plates that are arranged like stacks and spaced some distance apart, whereby
a) The plates are separated by spacers and are sealed off from one another in a gas-tight manner,
b) The plates are profiled in the shape of waves, so that flow channels are formed by the wave troughs that are separated from one another by the wave crests (fins),
c) The flow channels run parallel to one another and parallel to one side of the plate,
d) The flow channels at least partially contain at least one catalyst material that is introduced such that gaseous and/or liquid media can flow through the flow channels, and
e) The compact reactor has means (headers) for feeding at least two gaseous and/or liquid media to the flow channels or removing them therefrom.

The invention also relates to the use of a compact reactor and a process in this connection. The invention is described in the example of a process for the production of longer-chain hydrocarbons from methane, and a compact reactor that is used in this case for simultaneous implementation of endothermic vapor reforming and exothermic catalytic combustion, without being limited thereto. The compact reactor according to the invention is primarily suitable for implementing any endothermic and/or exothermic reactions.

A process for converting methane into longer-chain hydrocarbon is described in the patent publication WO2007/125360, the disclosure of which is hereby incorporated by reference. Such processes are essentially based on two catalytic reactions. First, a methane-containing feedstock is sent into a process for catalytic steam reforming. Corresponding to the reaction equation

CH₄+H₂O→CO+3H₂,

the methane of the feedstock is converted into synthesis gas. This reaction is endothermic. The necessary heat for the reaction is supplied by a catalytic combustion according to the prior art. The catalytic steam reforming starts only at a temperature of 400° C. Usually, the feedstocks for the catalytic combustion reaction are sent at a temperature of about 450° C. into the process for catalytic combustion and leave the latter at a starting temperature of between 800° C. and 850° C.

The synthesis gas-containing reaction products of catalytic steam reforming are sent as feedstock into a process for Fischer-Tropsch synthesis.

Corresponding to the reaction equation

nCO+2nH₂→(CH₂)n+nH₂O,

longer-chain hydrocarbons are formed from the synthesis gas. This reaction also runs on a catalyst material, but is exothermic in a temperature range of between 190° C. and 280° C. For an optimum plot of the reaction of the exothermic Fischer-Tropsch synthesis, the temperature has to be kept approximately constant so that the reaction according to the prior art is implemented in heat exchange with a coolant.

According to the prior art, both reactions are implemented in a compact reactor. Compact reactors for simultaneous implementation of steam reforming and heat-supplying catalytic combustion are described in both WO2007/129108 and EP1248675 (WO 01/51194), the disclosures of which are hereby incorporated by reference.

The compact reactor described in EP1248675 consists of a number of plates that are arranged like stacks and separated from one another. The plates are separated from one another by spacers and are sealed off from one another in a gastight manner. The feedstocks for the catalytic steam reforming and catalytic combustion are alternately distributed to the plates via means (headers) for feeding and removing. The plates are profiled in the shape of waves, whereby flow channels for the feedstocks of the respective reaction are formed by the wave troughs. The flow channels are separated from another by the wave crests (fins). The width of the wave trough is considerably larger here than the width of the fins. The flow channels run parallel to one another and parallel to one side of the plate. The flow channels of two adjacent plates, through which, on the one hand, the feedstock of the catalytic steam reforming is sent, and, on the other hand, the feedstocks of the catalytic combustion are sent, run perpendicular to one another. By the gastight seal between the respective plates, pressure and temperature of the media can be clearly distinguished in the flow channels of adjacent plates. The catalyst material is introduced into the flow channels, such that the flow of the media is maintained. Here, EP1248675 discloses metal foils that are made of aluminum-containing ferrite steel and that are arranged like waves, whereby, when heated in air, the ferrite steel forms an adhesive oxide coating made of aluminum oxide. The catalyst material is applied on the surface of the metal foils as well as on the surface of the flow channels. The wave density of the metal foils as well as the width of the flow channels can vary over the length in the direction of flow.

WO2007/129108 discloses a similar compact reactor with catalyst-bearing metal foils that are introduced in the form of waves in the flow channels and that have two different wave densities. Honeycomb-shaped structures with catalyst material on the surface in the flow channels are also disclosed.

In the compact reactors according to the prior art, the reactants in the feedstocks of the respective reactions are bypassed by the flow channels to the catalyst material. For an optimum reaction yield, preferably 100% of the reactants have to be in contact with the catalyst material for a sufficiently long time. This cannot always be achieved in the compact reactors according to the prior art. The dimensions of the plates and thus the length of the flow channels is relatively limited (EP1248675 quadratic plates with a 200 mm side length; WO2007/129108 rectangular plates with a 600 mm width and 1400 mm length). To achieve sufficient contact of all reactants with the catalyst material, a good thorough mixing of the reactants in the flow channels thus has to be ensured.

An aspect of this invention is therefore to improve the thorough mixing of the reactants of the gaseous and/or liquid media in a compact reactor comprising: a number of stacked plates that are spaced some distance apart, wherein
a) The plates are separated by spacers and are sealed off from one another in a gas-tight manner,
b) The plates are profiled in the shape of waves, so that flow channels are formed by the wave troughs that are separated from one another by the wave crests (fins),
c) The flow channels run parallel to one another and parallel to one side of the plate,
d) The flow channels at least partially contain at least one catalyst material that is introduced such that gaseous and/or liquid media can flow through the flow channels, and
e) The compact reactor has means (headers) for feeding at least two gaseous and/or liquid media to the flow channels or removing them therefrom.

In accordance with the invention, a compact reactor of the above-mentioned type is provided wherein the reactor possesses means whereby the gaseous and/or liquid medium that flows in the flow channels can flow through the fins (wave crests) between the individual flow channels of a plate.

### BRIEF DESCRIPTION OF THE DRAWINGS

Various other features and attendant advantages of the present invention will be more fully appreciated as the same becomes better understood when considered in conjunction with the accompanying drawings, in which like reference characters designate the same or similar parts throughout the several views, and wherein:

FIG. 1 illustrates an arrangement of stacked plates for use in a compact reactor of the above-mentioned type as described in FIG. 1 of WO 2007/129108; and

FIG. 2 illustrates an overall embodiment of a compact reactor of the above-mentioned type as described in FIG. 2 of WO 2007/129108.

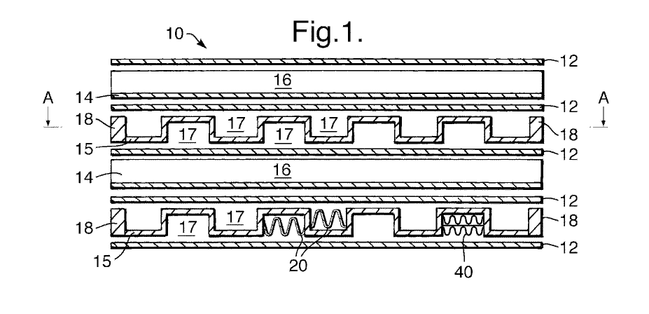

As described in WO 2007/129108, FIG. 1 illustrates a reactor 10 suitable for use as a steam reforming reactor. The reactor 10 comprises a stack of plates that are rectangular in plan view, each plate being of corrosion resistant high-temperature alloy such as Inconel 625, Incoloy 800HT or Haynes HR-120. Flat plates 12, typically of thickness in the range 0.5 to 4 mm, in this case 1 mm thick are arranged alternately with castellated plates 14, 15 in which the castellations are such as to define straight-through channels 16, 17 from one side of the plate to the other. The castellated plates 14 and 15 are arranged in the stack alternately, so the channels 16, 17 are oriented in orthogonal directions in alternate castellated plates. The thickness of the castellated plates 14 and 15 (typically in the range between 0.2 and 3.5 mm) is in each case 0.75 mm. The height of the castellations (typically in the range 2-10 mm) is 4 mm in this example, and solid bars 18 of the same thickness are provided along the sides. In the castellated plates 15 which define the combustion channels 17 the wavelength of the castellations is such that successive ligaments are 25 mm apart, while in the castellated plates 14 which define the reforming channels 16 successive ligaments are 15 mm apart.

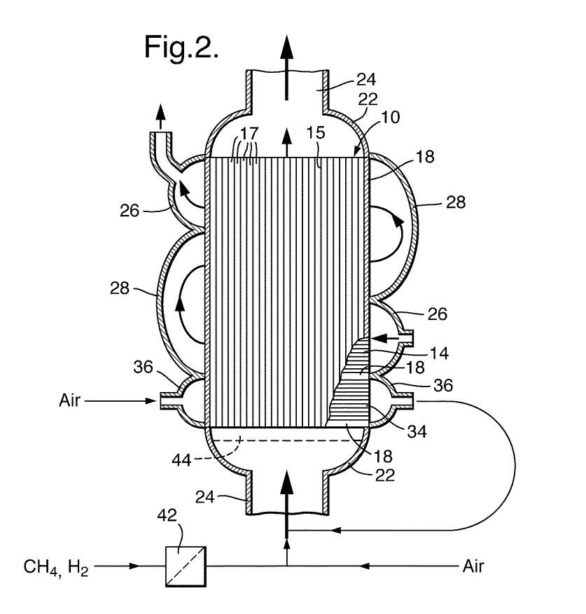

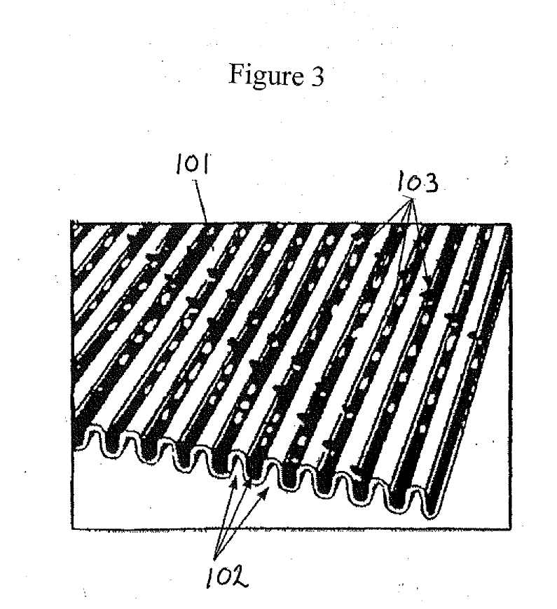

As described in WO 2007/129108, FIG. 2 illustrates a sectional view through the assembled reactor 10, each plate 12 is rectangular, of width 600 mm and of length 1400 mm; the section is in a plane parallel to one such plate 12. The castellated plates 15 for the combustion channels 17 are of the same area in plan, the castellations running lengthwise. The castellated plates 14 for the reforming channels 16 are 600 mm by 400 mm, three such plates 14 being laid side-by-side, with edge strips 18 between them, with the channels 16 running transversely; a castellated plate 34 with identical castellations, 600 mm by 200 mm in plan, is laid side-by-side with one of the plates 14.

Headers 22 at each end of the stack enable the combustion gases to be supplied to, and the exhaust gases removed from, the combustion channels 17 through pipes 24. Small headers 26 (bottom right and top left as shown) enable the gas mixture for the reforming reaction to be supplied to the channels 16 in the first of the castellated plates 14, and the resulting mixture to be removed from those in the third castellated plate 14; double-width headers 28 (top right and bottom left as shown) enable the gas mixture to flow from one castellated plate 14 to the next. Separate small headers 36 communicate with the channels defined by the plates 34. The overall result is that the gases undergoing reforming follow a serpentine path that is generally co-current relative to the flow through the combustion channels 17.

The stack is assembled as described above, and bonded together typically by diffusion bonding, brazing, or hot isostatic pressing. Corrugated metal foil catalyst carriers 20 (only two of which are shown, in FIG. 1) are then inserted into each of the channels 16 and 17, carrying catalysts for the two different reactions. The metal foil is covered with a ceramic coating containing the catalyst. In the reforming channels 16 (in the plates 14) the catalyst carriers 20 extend the entire length of the channel. In the combustion channels 17 the catalyst carriers 20 are of length 1200 mm, so that they extend alongside the reforming channels 16; the first 200 mm length of each channel 17 is instead occupied by a non-catalytic corrugated foil insert 40 (only one is shown, in FIG. 1) made of a stack of two corrugated foils and a flat foil, the wavelength of the corrugations being such that the flow paths are significantly smaller than those through the catalyst carriers 20, and in this case the foil is of stainless steel. After insertion of the catalyst carriers 20 and the non-catalytic inserts 40, the headers 22, 26, 28 and 36 are attached to the outside of the stack, as shown in FIG. 2. The catalyst carriers 20 and the non-catalytic inserts 40 are not shown in FIG. 2, and are shown only diagrammatically in FIG. 1.

The basic idea of the invention is to improve the thorough mixing of the individual reactants in the gaseous and/or liquid media in the individual flow channels by a cross-mixing between the individual flow channels. The same gaseous and/or liquid medium flows into the flow channels of one plate. In accordance with the invention, cross-mixing between the individual flow channels and thus also the thorough mixing of the reactants in the individual flow channels is improved by the permeability of the fins to this medium. As a result, the contact of the reactants with the catalyst material and consequently the reaction sequence or the reaction yield are improved.

In addition, the entire temperature profile of the compact reactor is improved and becomes considerably more uniform by the permeability of the fins according to the invention. At the locations where the respective medium can flow through the fins, increased turbulence in the flow of media is formed by the cross-mixing that is made possible thereby. By the higher turbulence, the heat transfer from the flowing media to the fins and thus to the plates is improved. As a result, the heat exchange between media separated by the plates is considerably improved, and a more homogeneous and more uniform temperature profile in the compact reactor is formed.

By the permeability of the fins according to the invention, the cross-distribution of the media within one flow plane and, at the same time, the temperature transition between two adjacent flow planes are therefore both considerably improved. The width of the fins and the flow channels in this case can be thoroughly different and can be optimized in the reactions that are occurring in each case.

In a preferred embodiment of the invention, the fins are perforated on the walls. The perforation of the fins is a simple method to make the gaseous and/or liquid medium flow through the fins.

According to an especially preferred design of the invention, the catalyst material is introduced into the flow channels of the compact reactor in the form of a corrugated foil, whereby the foil is perforated. Catalyst material in the form of foils that are arranged like waves is already known in the prior art, and is an established means for contacting the reactants with the catalyst material without greatly impairing the flow of the reactants. By the advantageous perforation of the foil, a further improvement of the cross-mixing with the associated, already described advantages is achieved.

According to a further aspect of the invention, the compact reactor according to the invention is used for simultaneous implementation of endothermic steam reforming and catalytic combustion or for implementing Fischer-Tropsch synthesis in heat exchange with a coolant.

Performing a process for simultaneous implementation of endothermic steam reforming and catalytic combustion in a compact reactor according to the invention at a temperature of 700° C.-850° C., especially preferably below 750° C., also proves advantageous. In an especially preferred configuration of the invention, a starting temperature of the media from the compact reactor of 750° C. is not exceeded in the catalytic combustion reaction.

With this invention, it is possible in particular to improve thorough mixing of the reactants in the gaseous and/or liquid medium within the flow channels of the compact reactor. As a result, an improved reaction scheme is achieved.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding German application No. 10 2008 017 342.8, filed Apr. 4, 2008.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compact reactor comprising: a number of plates arranged in a stack and spaced apart from each other, wherein
a) said plates are separated by spacers and are sealed off from one another in a gas-tight manner,
b) each of said plates are profiled in the shape of waves, so that flow channels are formed by the wave troughs that are separated from one another by the wave crests (fins),
c) said flow channels run parallel to one another and parallel to one side of the plate,
d) said flow channels at least partially contain at least one catalyst material that is introduced such that gaseous and/or liquid media can flow through the flow channels,
e) said compact reactor has means (headers) for feeding at least two gaseous and/or liquid media to the flow channels or removing them therefrom, and
f) the gaseous and/or liquid medium that flows in the flow channels can flow through the fins between the individual flow channels of a plate.

2. A compact reactor according to claim 1, wherein said fins are perforated on their walls.

3. A compact reactor according to claim 1, wherein said catalyst material is introduced into the flow channels in the form of a corrugated foil, and said foil is perforated.

4. A compact reactor according to claim 2, wherein said catalyst material is introduced into the flow channels in the form of a corrugated foil, and said foil is perforated.

5. In a process of performing endothermic steam reforming and catalytic combustion, the improvement endothermic steam reforming and catalytic combustion are simultaneously implemented in a compact reactor according to claim 1.

6. In a process of performing a Fischer-Tropsch synthesis in heat exchange with a coolant, the improvement wherein said Fischer-Tropsch synthesis is performed in a compact reactor according to claim 1.

7. A process according to claim 5, wherein said process is implemented in a temperature range of between 700° C. and 850° C.

8. A process according to claim 7, wherein said process is implemented in a temperature below 750° C.
